(19) United States
(12) Patent Application Publication
(10) Pub. No.: US 2011/0288115 A1
(43) Pub. Date: Nov. 24, 2011

(54) TREATMENT OF VAGALLY-MEDIATED SPECTRUM DISORDERS

(75) Inventor: Peter Kelbe, Sugar Grove, IL (US)

(73) Assignee: AVMEDIS LLC, Sugar Grove, IL (US)

(21) Appl. No.: 13/040,122

(22) Filed: Mar. 3, 2011

Related U.S. Application Data
(60) Provisional application No. 61/347,601, filed on May 24, 2010.

(51) Int. Cl. A61K 31/438; A61P 9/12; A61P 1/04; A61P 25/30; A61K 31/427; A61P 25/28; A61P 25/22; A61P 25/18; A61P 25/24; A61P 11/06; A61P 3/04

(52) U.S. Cl. 514/299; 514/365

(57) ABSTRACT

The present invention provides a method of treatment of a vagally-mediated spectrum disorder, comprising administering an effective amount of a 5-HT₃ receptor antagonist to a patient in need thereof.

### CROSS-REFERENCES TO RELATED APPLICATIONS

The Present Application claims the benefit of U.S. Provisional Patent Application No. 61/347,601, filed May 24, 2010. The content of this U.S. Provisional Patent Application is hereby incorporated herein in its entirety.

### BACKGROUND

The vagus nerve, also called pneumogastric nerve, cranial nerve X, the Wanderer or sometimes the Rambler, is the tenth of twelve (excluding CNO) paired cranial nerves, that emerge directly from the brain, in contrast to spinal nerves which emerge from segments of the spinal cord. Upon leaving the medulla between the olivary nucleus and the inferior cerebellar penduncle, the vagus nerve extends through the jugular foramen, then passes into the carotid sheath between the internal carotid artery and the internal jugular vein down below the head, to the neck, chest and abdomen, where it contributes to the innervation of the viscera.

Besides output to the various organs in the body, the vagus nerve conveys sensory information about the state of the body's organs to the central nervous system (CNS). 80-90% of the nerve fibers in the vagus nerve are “afferent” (sensory) nerves communicating the state of the viscera to the brain [1]. Sometimes the branches of the vagus are spoken of in the plural and are thus called “vagi.” The vagus is also called the pneumogastric nerve since it innervates both the lungs and the stomach.

In part, vagus nerve function is mediated by 5-HT receptors. In particular, the 5-HT₃ receptor is the only serotonin receptor which is a ligand gated ion channel, thus regulating ion flux across the neuronal membrane. It therefore regulates resting membrane potential and release of transmitters such as neurotransmitters. The primary effects of 5-HT₃ receptors appear to be peripheral as their distribution is heavily concentrated on the afferent fibers of the vagus nerve, whereas there appear to be relatively few 5-HT₃ receptors within the CNS.

The 5-HT₃ receptor includes 5 subunits arranged around a ligand gated ion channel, which is permeable to sodium, potassium, and calcium ions. Binding of the neurotransmitter 5-hydroxytryptamine (serotonin) to the 5-HT₃ receptor opens the channel, giving rise to an ion flux which, in turn, leads to an excitatory response in neurons. This response can regulate the release and production of transmitters. Activation of the ligand gated ion channel by an agonist results in an influx of calcium which leads to the migration of transmitter-containing vesicles to the cell membrane and the release of the vesicle contents into the synaptic cleft at the terminus during the excitatory response (or into the surrounding tissues if release occurs along the nerve cell processes). By contrast, antagonists inhibit the flow of cations, calcium in particular, through the channel, thus inhibiting the release of transmitters in a dose- and time-dependent fashion.

5-HT₃ receptor antagonists include a defined and recognized class of pharmaceutically active compounds, well known in the art and characterized by their pharmacological activity. For example, suitable 5-HT₃ receptor antagonists are described in U.S. Pat. Nos. 5,225,407 and 5,298,510. In addition, several 5-HT₃ receptor antagonists are commercially available and have been approved by the FDA for treating and preventing chemotherapy and/or radiation-induced nausea and vomiting, and for controlling post-operative nausea and vomiting.

It has now been found that compounds that act as antagonists of 5-HT₃ receptor may be useful for the treatment of asthma, essential hypertension, gastroesophageal reflux disease (GERD), substance abuse induction, substance abuse withdrawal, satiety disorders, obesity, chronic pain (for example due to fibromyalgia), secondary symptoms of Alzheimer's disease, age-associated memory impairment, and somatic symptoms associated with psychiatric disorders, such as: anxiety and panic disorders, post-traumatic stress disorder (PTSD), impulse control disorders, depression, and psychoses.

### SUMMARY

In a first set of representative embodiments, the present invention provides a method of treating a vagally-mediated spectrum disorder comprising administering to a patient in need of such treatment, an effective dose of a 5-HT₃ receptor antagonist.

In a second set of representative embodiments, the present invention provides a method of treating a vagally-mediated spectrum disorder by administering a compound that antagonizes 5-HT₃ receptor activity, thereby preventing or inhibiting abnormal afferent vagal nerve signaling.

In a third set of representative embodiments, the present invention provides a method of treatment of a panic disorder, comprising administering an effective amount of a 5-HT₃ receptor antagonist to a patient in need thereof.

In a fourth set of representative embodiments, the present invention provides a method of treatment of substance abuse, comprising administering an effective amount of a 5-HT₃ receptor antagonist to a patient in need thereof.

In a fifth set of representative embodiments, the present invention provides a method of treatment of post-traumatic stress disorder, comprising administering an effective amount of a 5-HT₃ receptor antagonist to a patient in need thereof.

### DEFINITIONS

A transmitter is a molecule which relays, amplifies, and modulates signals between two or more cells. Example transmitters include neurokinins, neurotrophins, chemokines, cytokines, other pro-inflammatory mediators, and neurotransmitters.

“Treatment” as used herein includes the alleviation, amelioration or control of a pathology, disease, disorder, process, condition or event. In this context, the term “treatment” is further to be understood as embracing use to reverse, restrict or control progression of any specified pathology, disease, disorder, process, condition, or event. If any pathology, disease, disorder, process, condition or event is associated with pain, the term “treatment” preferably encompasses the alleviation, amelioration or control (including temporal or permanent removal) of at least one further sequela or symptom in addition to pain, such as inflammation, cough, effusion, anxiety, and more preferably of all symptoms and most preferably of the total clinical picture of the respective pathology, disease, disorder, process, condition or event.

Asthma is a respiratory disease that involves inflammation and narrowing of the airways leading to the lungs. Asthma is characterized by sudden recurring attacks of labored breathing, chest constriction, and coughing.

“Substance abuse” refers to a maladaptive pattern of use of a substance. Some of the substances most often associated with this term include alcohol, amphetamines, barbiturates, benzodiazepines, cocaine, methaqualone, and opioids.

“Substance abuse withdrawal” as used herein refers to the group of symptoms that occurs upon the abrupt discontinuation/separation or a decrease in dosage of the intake of substances such as medications, recreational drugs, and alcohol.

“Substance abuse induction” as used herein refers to the induction of substance dependence due to the intake of substances such as medications, recreational drugs, and alcohol.

“Panic disorder” is an anxiety disorder characterized by recurring severe panic attacks. It may also include significant behavioral change lasting at least a month and of ongoing worry about the implications or concern about having other attacks. The latter are called anticipatory attacks (DSM-IVR).

Secondary symptoms of Alzheimer's disease include depression, agitation, sleep disorders, dementia, forced eyelid closure, speech problems, drooling, difficulty swallowing, constipation, breathing problems, difficulty voiding, dizziness, stooped posture, swelling of the feet, and sexual problems.

“Chronic pain” as used herein refers to pain manifesting itself for longer than three months after the onset of pain and includes, for example, the pain associated with fibromyalgia.

“Satiety disorders” as used herein refers to conditions featuring the partial or total loss of satiety. Such disorders are usually associated with hyperphagia (overeating) which can in turn lead to obesity.

The term “post-traumatic stress disorder,” or PTSD, as used herein refers to an anxiety disorder that can develop after exposure to an event which results in psychological trauma.

The term “vagally-mediated spectrum disorder” or VMSD is used herein to refer to a class of diseases and/or conditions whose cause, continuance, and/or progression is mediated by aberrant afferent vagal nerve signaling.

### DETAILED DESCRIPTION

It has now been found that compounds that act as antagonists of 5-HT at 5-HT3 receptors may be useful for the treatment of a spectrum of disorders referred to herein as vagally-mediated spectrum disorders (VMSDs).

The present invention provides a new medical use for 5-HT₃ receptor antagonists and pharmaceutical compositions containing them. In particular, the invention relates to the use of 5-HT₃ receptor antagonists in appropriate dosings and regimens for the treatment of pathologies such as asthma, essential hypertension, gastroesophageal reflux disease (GERD), substance abuse induction, substance abuse withdrawal, satiety disorders, obesity, chronic pain (for example due to fibromyalgia), secondary symptoms of Alzheimer's disease, age-associated memory impairment, and somatic symptoms associated with psychiatric disorders, such as anxiety and panic disorders, post-traumatic stress disorder, impulse control disorders, depression, and psychoses.

In one aspect, the present invention provides a method for inhibiting or stopping abnormal signaling by the afferent vagal nerve comprising administering to a patient in need of treatment an effective dose of a 5-HT₃ receptor antagonist. In this aspect, the present method is expected to halt the anterograde transport of one or more transmitters into the CNS, and to downregulate the transcription of one or more genes encoding transmitters in the nerve cells. Exemplary transmitters include neurokinins, neutrophins, chemokines, cytokines, other pro-inflammatory mediators, and neurotransmitters.

In another aspect, the present invention provides a method of treating a patient suffering from one or more of the following diseases/conditions: asthma, essential hypertension, gastroesophageal reflux disease (GERD), substance abuse induction, substance abuse withdrawal, satiety disorders, obesity, chronic pain (for example due to fibromyalgia), secondary symptoms of Alzheimer's disease, age-associated memory impairment, and somatic symptoms associated with psychiatric disorders, such as: anxiety and panic disorders, post-traumatic stress disorder, impulse control disorders, depression, and psychoses, comprising administering an effective dose of a 5-HT₃ receptor antagonist.

The present invention is based on the discovery that abnormal vagal afferent signaling mediates a number of pathologies in a variety of tissues and organs by a mechanism involving the CNS. Without being bound to any particular theory, it is believed that abnormal signaling from afferent vagal nerves underlies at least partially the mechanism for the initiation and/or progression of a class of disorders referred to herein as VMSDs. It is believed that some type of triggering event(s), for example, an allergic response, trauma, substance abuse, infection or other pathologies in one or more organs and/or tissues, results in the production and anterograde movement of transmitters, such as neurokinins, neutrophins, chemokines, cytokines, and neurotransmitters from the nerve cell bodies in the ganglia associated with the afferent vagal nerves through the axons of the afferent vagal nerves, thus bypassing the choroid plexus (also known as the blood brain barrier). The transmitters are then released within the CNS at the synapses located in the midbrain and cortex. Such chemicals have the ability to alter the structure and function of post-synaptic neurons in a dose- and time-dependent fashion. By this mechanism it is believed that peripheral parasympathetic signaling influences the CNS and sympathetic output to cause pathologies in tissues, organs, and the brain.

Again, without being bound to any particular theory, it is further believed that the chemicals move retrograde from the nerve cell bodies in the ganglia associated with the afferent vagal nerves through the neuronal processes back to the tissues, where they can produce pathologic effects, thereby exacerbating the pathologic effects due to the afferent response, or can be stored awaiting a future stimulus or insult for release. In this manner, it is believed that many diverse and seemingly unrelated pathologies, such as those listed above, share a common underlying mechanism, or etiology, thereby belonging to the class of disorders referred to herein as “vagally-mediated spectrum disorders (VMSDs).”

The present invention provides a method for treatment of vagally-mediated spectrum disorders by administration of an effective amount of at least one 5-HT₃ receptor antagonist to a patient in need thereof. The term “vagally-mediated spectrum disorders” means any disease or other deleterious condition or state in which abnormal vagal afferent signaling plays a mechanistic role e.g. in initiating and/or propagating the disease or condition. Such disorders include, without limitation, asthma, essential hypertension, gastroesophageal reflux disease (GERD), substance abuse induction, substance abuse withdrawal, satiety disorders, obesity, chronic pain (for example due to fibromyalgia), secondary symptoms of Alzheimer's disease, age-associated memory impairment, and somatic symptoms associated with psychiatric disorders, such as: anxiety and panic disorders, post-traumatic stress disorder, impulse control disorders, depression, and psychoses.

Any 5-HT₃ receptor antagonist can be used in accordance with the invention as disclosed, for example, in U.S. Pat. Nos. 5,225,407 and 5,298,510. Preferred 5-HT₃ receptor antagonists which may be employed in accordance with the present invention are: (A) ondansetron [1,2,3,9-tetrahydro-9-methyl-3-[(2-methyl-1H-imidazol-1-yl]methyl]-4H-carbazol-4-one; (B) granisetron [endo-1-methyl-N-(9-methyl-9-aza-bicyclo [3.3.1]non-3-yl)-1 H-imidazole-3-carboxamide; (C) dolasetron [1 H-indole-3-carboxylic acid (2a 6a, 8a, 9a(3)-octahydro-3-oxo-2,6-methano-2H-quinolizin-8-yl ester]; (D) tropisetron [(Indol-3-yl-carboxylic acid-endo-8-methyl-8-aza-bicyclo[3,2,1]-oct-3-yl-ester)]; (E) ramosetron 8 4,5,6,7-tetrahydro-5-[(1-methyl-indol-3-yl)carbonyl]65 benzimidazole]; (F) fabesetron [(+)-10-methyl-7-(5-methyl-1H-imidazol-4-ylmethyl)-6,7,8,9-tetrahydropyrido[1,2-a]indol-6-one]; (G) lintopride [N-(1-ethyl-2-imidazolin-2-yl-methyl)-2-methoxy-4amino-5-chlorobenzamide]; and (H) alosetron [2,3,4,5-tetrahydro-5-methyl-2-[(5-methyl-1H-imidazol-4-yl)methyl]-1H-pyrido[4,3-b]indol-1-one].

5-HT₃ receptor antagonists may be employed in accordance with the invention in free or in pharmaceutically acceptable salt form, e.g. as known in the art, for example, in the case of compounds (A) to (D) above in pharmaceutically acceptable acid addition salt form. Pharmaceutically acceptable salts include, in the case of: compound (A) the hydrochloride dihydrate; compound (B) the hydrochloride; compound (C) the mesylate; and compound (D) the monohydrochloride. References to 5-HT₃ receptor antagonists collectively or individually throughout the present specification and claims are accordingly to be understood as embracing both free compounds and such pharmaceutically acceptable salt forms, e.g. as clinically employed, and further also solvates, e.g. hydrates, or specific crystal forms of any of these compounds or salts.

For use in accordance with the present invention the appropriate dosage will, of course, vary depending on, for example, the particular 5-HT₃ receptor antagonist employed, the mode of administration, and the nature and severity of the condition to be treated as well as the specific condition to be treated and is within the purview of the treating physician. An indicated daily dosage will be in the range usually employed for known indications such as emesis and will typically be from about 0.05 mg to about 50 mg per day, conveniently administered once daily, or in divided doses up to four times a day or in sustained release form. In the case of ondansetron, an appropriate dosage for administration will usually be in the range of about 8 mg to about 30 mg per day, administered once daily, for an interval, for example, of 5 to 10 days, or 5 to 30 days, or also 5 to 60 days.

For use in accordance with the present invention, 5-HT₃ receptor antagonists may be administered by any conventional route, in particular: enterally, orally, nasally, e.g. in the form of tablets or capsules, via suppositories, or parenterally, e.g. in the form of injectable solutions or suspensions, for intravenous, intra-muscular, sub-cutaneous, or intra-peritoneal injection. Administration of granisetron via nasal spray is particularly preferred.

Suitable formulations and pharmaceutical compositions for use in accordance with the present invention will include those formulated in a conventional manner using one or more physiologically acceptable carriers or excipients, and any of those known and commercially available and currently employed in the clinical setting. Thus, the compounds for use accordance to the present invention may be formulated for oral, buccal, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound over an extended period.

For buccal administration the compositions may take the form of tablets or lozenges formulated in a conventional manner known to the skilled artisan.

Compounds for use according to the present invention may also be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the active ingredient may be in powder form for constitution with a suitable vehicle, e.g. sterile pyrogen-free water, before use.

The compounds for use according to the present invention may also be formulated for rectal administration as suppositories or retention enemas, e.g. containing conventional suppository bases such as cocoa butter or other glycerides.

The invention has been described with reference to various illustrative embodiments and techniques. However, it should be understood that many variations and modifications, as are known in the art, may be made while remaining within the scope of the claimed invention. The examples that follow are illustrative and are not intended to be limiting.

### EXAMPLE 1

### Treatment of Panic Disorder with 5-HT₃ Receptor Antagonist

A patient presents with symptoms including rapid heartbeat, perspiration, dizziness, dyspnea, trembling, uncontrollable fear, and hyperventilation, indicative of panic disorder. The patient has previously been treated, largely unsuccessfully, with selective serotonin reuptake inhibitors (SSRIs), monoamine oxidase inhibitors (MAOIs), tricyclic antidepressants, and benzodiazepines.

The patient is treated for 5 to 60 days sequentially with ondansetron administered at a dose of 8 mg/day to 30 mg/day. Treatment results in long-lasting amelioration of panic disorder with a reduction in the severity and frequency of panic attacks and their symptoms.

### EXAMPLE 2

### Treatment of Alcohol Abuse

The patient is a 53 year old female suffering from alcohol dependence and complaining of symptoms including abdominal pain, confusion, nausea and vomiting, neglecting to eat, and shaking in the morning. The patient is prescribed granisetron 5 mg per day taken via nasal spray. Within one month the patient reports that all of the above symptoms have improved considerably and that she is now able to exert control over her drinking.

Equivalent results are obtainable in equivalent or comparable trials with patients exhibiting similar symptomatology by employing 5-HT₃ receptor antagonists other than ondansetron, for example by using granisetron, dolasetron, tropisetron, ramosetron, fabesetron, lintopride, and alosetron, at comparable, e.g. conventional clinical doses as known in the art. Similar results are also achievable employing 5-HT₃ receptor antagonists in clinical trials involving subjects exhibiting other vagally-mediated spectrum disorders.

### REFERENCES

[1] Fuller G N, Burger P C. “Nervus terminalis (cranial nerve zero) in the adult human.” Clin Neuropathol 9, no. 6 (Nov-Dec 1990): 279-283.

1. A method of treatment of a vagally-mediated spectrum disorder, comprising administering an effective amount of a 5-HT₃ receptor antagonist to a patient in need thereof.

2. The method of claim 1, wherein the vagally-mediated spectrum disorder is selected from the group consisting of asthma, essential hypertension, gastroesophageal reflux disease, substance abuse induction, substance abuse withdrawal, satiety disorders, obesity, chronic pain, secondary symptoms of Alzheimer's disease, age-associated memory impairment, anxiety disorders, panic disorders, post-traumatic stress disorder, impulse control disorders, depression, psychoses, and combinations thereof.

3. The method of claim 2, wherein the vagally-mediated spectrum disorder is selected from the group consisting of substance abuse induction, substance abuse withdrawal, panic disorder, post-traumatic stress disorder, and combinations thereof.

4. The method of claim 1, wherein the 5-HT₃ receptor antagonist is selected from the group consisting of ondansetron, granisetron, dolasetron, tropisetron, ramosetron, fabesetron, lintopride, alosetron, and combinations thereof.

5. The method of claim 4, wherein the 5-HT₃ receptor antagonist is ondansetron.

6. The method of claim 1, wherein administering of said 5-HT₃ receptor antagonist is by a mode selected from the group consisting of: enterally, orally, nasally, suppositories, parenterally, or a combination thereof.

7. The method of claim 1, wherein administering the 5-HT₃ receptor antagonist to the patient is by means of a nasal spray.

8. The method of claim 1, wherein the 5-HT₃ receptor antagonist is administered daily in an amount from about 0.05 mg to about 50 mg.

9. The method of claim 1, wherein the amount of 5-HT₃ receptor antagonist is from about 8 mg per day to about 30 mg per day.

10. The method of claim 1, further comprising administering the 5-HT₃ receptor antagonist for at least one interval of 5 to 60 days.

11. The method of claim 1, further comprising administering the 5-HT₃ receptor antagonist for at least one interval of 5 to 30 days.

12. The method of claim 1, further comprising administering the 5-HT₃ receptor antagonist for at least one interval of 5 to 10 days.

13. The method of claim 1, wherein at least one sequela or symptom that is associated with the vagally-mediated spectrum disorder is alleviated, ameliorated or controlled.

14. The method of claim 13, wherein the at least one sequela or symptom is selected from the group consisting of rapid heartbeat, perspiration, dizziness, dyspnea, trembling, uncontrollable fear, hyperventilation, abdominal pain, confusion, nausea, vomiting, neglecting to eat, shaking in the morning, and combinations thereof.

15. A method of treatment of a panic disorder, comprising administering an effective amount of a 5-HT₃ receptor antagonist to a patient in need thereof.

16. The method of claim 15, wherein the 5-HT₃ receptor antagonist is ondansetron.

17. The method of claim 15, further comprising administering the 5-HT₃ receptor antagonist for at least one interval of 5 to 60 days.

18. A method of treatment of substance abuse withdrawal, comprising administering an effective amount of a 5-HT₃ receptor antagonist to a patient in need thereof.

19. The method of claim 18, wherein the 5-HT₃ receptor antagonist is granisetron.

20. The method of claim 18, further comprising administering the 5-HT₃ receptor antagonist for at least one interval of 5 to 60 days.
